(19) United States
(12) Patent Application Publication
(10) Pub. No.: US 2014/0193531 A1
(43) Pub. Date: Jul. 10, 2014

(54) Aqueous extracts of deschampsia antarctica

(71) Applicant: Gidekel Manuel, Santiago (CL)

(72) Inventor: Gidekel Manuel, Santiago (CL)

(73) Assignee: Uxmal S.A

(21) Appl. No.: 13/694,380

(22) Filed: Nov. 27, 2012

(51) Int. Cl. A61K 36/899; A61Q 19/08; A61K 8/97

(52) U.S. Cl. CPC A61K 36/899; A61K 8/97; A61Q 19/08
USPC 424/750

(57) ABSTRACT

The present invention encompasses a method of preparing a pharmaceutically active extract of D. Antarctica comprising the steps of:
a. Collecting and disinfecting D. Antarctica plants;
b. Exposing the D. Antarctica plants to ultra pure water at a temperature of from 95° to 100° C. for a period of about 5 hours to yield a liquid extract of the plant; and
c. Lyophilizing the liquid extract yielding a dry homogeneous product.

In another embodiment, the present invention provides an anti-aging pharmaceutical composition useful in slowing down or reversing the aging process in human beings said composition comprising an aqueous extract of D. Antarctica. In a particular embodiment the aqueous extract comprises a polyphenolic free-radical scavenger present in the amount of at least 4.0% (w/w) and preferably at least 5.0% (w/w)

In another embodiment the present invention provides a method of treatment for the prevention of photoaging effects generated by exposure to harmful ultraviolet radiation comprising the step of administering to a human being a therapeutically effective amount of the aqueous extract of D. Antarctica. Preferably the extract is administered in the form of a cream, paste or gel

### FIELD OF THE INVENTION

The present invention relates to methods of preparing aqueous extracts of D. antarctica having anti-oxidant and anti-inflammatory properties as well as to pharmaceutical compositions comprising aqueous extracts of D. antarctica at concentrations useful in the prevention of aging in humans as well as for the prevention of photoaging effects generated by exposure to harmful ultraviolet radiation.

Genes and functional enrichments are identified as factors relating the therapeutic effects of the extract in the prevention of photoaging and cancer induced by ultraviolet radiation.

### BACKGROUND OF THE INVENTION

The continent of Antarctica is considered one of the world's most primitive ecosystems, with extreme environmental conditions. It is covered year-round by ice and snow, so plants grow on only 2% of the land. Deschampsia antarctica Desv. is a vascular angiosperm from the Poaceae family that has naturally colonized maritime Antarctica (Lewis Smith, 2003). The species is physiologically and biochemically adapted to the action of different abiotic factors such as high and low radiation, low precipitation, drought, flooding, salinity, and extremely low temperatures that sometimes come with frosts, ice, and snow (Alberdi et al., 2002; Barcikowski A et al., 1999; Bravo et al., 2001; Bravo and Griffith, 2005; Bystrzejewska, 2001; Day et al., 2001; Lewis Smith, 2003; Zuñiga et al., 1996)

D. antarctica can usually be found on the South Orkney Islands and in maritime Antarctica south of 68° S latitude, without extending onto the continent of Antarctica itself (Alberdi et al., 2002). Its growth period starts in November (springtime) and spreads through seed germination or through tillers sprouting from previous years' plants (Corte, 1961; Holderegger et al., 2003). The plant's sexual reproduction is scarce due to the fact that the climatic conditions limit the seeds' flowering and maturing, and it is unable to complete its reproductive cycle with gametes (Ruhland and Day, 2001). However, it is considered that D. antarctica colonizes new areas on the continent of Antarctica primarily through plant dispersal.

The skin is an organ highly susceptible to ultraviolet (UV) radiation from the sun. It is known that skin cancer is related to exposure to UV radiation. Chronic exposure to UV rays is associated with actinic keratosis, scaly cell cancer, and basal cell cancer; while intermittent, intensive exposure to UV radiation is connected with melanoma, the most harmful type of skin cancer. There are two types of UV radiation, known as UVA (320-400 nm) and UVB (280-320 nm). The later induces acute and chronic damage, primarily by damaging DNA. On the contrary, UVA rays penetrate the skin more deeply and indirectly damage DNA by producing reactive oxygen species (ROS). Ultraviolet rays are responsible for a number of other biological effects on the skin, including premature aging in the form of wrinkles, dryness, sagging, telangiectasia (blood vessels evident), dark patches, freckles, and pigmentation (Wang et al., 2010).

Ageing is a process of progressive decreases in the maximal functioning and reserve capacity of all organs in the body, including the skin. Photoageing is the superposition of the chronic ultraviolet (UV)-induced damage on intrinsic ageing and accounts for most age-associated changes in skin appearance. Although a substantial worldwide problem for millions of people and the stimulus for a $10 billion anti-ageing skin products market, photoaging is also important for its intimate relationship to photocarcinogenesis (Yaar and Gilchrest, 2007).

The photoaging process includes molecular and structural damage of the skin, such as inflammation, decreased synthesis of collagen, thickening or proliferation of the epidermis (upper layer of the skin) and incomplete degradation of collagen fragments. This is caused by receptors that initiate signaling mitochondrial damage, protein oxidation and DNA damage response based on telomerase (Yaar and Gilchrest, 2001).

The carcinogenic effects of ultraviolet radiation can be decreased through apoptosis, or programmed cell death, and the elimination of the damaged DNA or mutated cells. Microarrays genes expression studies have shown that UVA and UVB radiation causes expression changes in keratinocytes; these changes have led to a greater understanding of the impact of UV radiation on keratinocytes at the molecular level, especially the mechanism associated with apoptosis (He et al., 2004; Lee et al., 2005). Various changes at the molecular level have been associated with aging and cancer caused by high exposure to ultraviolet rays, as exposure to UV radiation triggers a stream of ROS and inflammatory keratinocytes by activating the AP-1 and NF-κB pathways.

The development of sunscreens that protect against both UVA and UVB radiation, as well as products that antagonize the signaling pathways leading to photoageing UV are important steps to prevent and reverse photoageing. A better understanding of the mechanism of skin UV protection also gives rise to novel treatments that promote not only the improvement of the appearance of the skin, but also reduce skin cancer.

Antarctic hair grass (Deschampsia antarctica desv; Poceae) is the only Gramineae that tolerates harsh stress conditions (wind, salinity, cold, light and UV radiation) present in the antarctic territory. A species that manages to grow and survive well in a hostile environment undoubtedly has to be well adapted to tolerate UV radiation. This characteristic is what makes Antarctic hair grass an ideal candidate for extracting new natural active compounds with beneficial biological properties and effects.

Recent works show that D. antarctica extract present photoprotective properties, which can be attributed to molecules, such as flavonoids and carotenoides, which act as UV-absorbing molecules and as antioxidants, as well as stimulate DNA-repair processes (Pereira et al., 2009). Photoprotective activity of the D. antarctica is something already known. In previous studies, the extract of this plant has been postulated as a new agent for skin photoprotection against UVA and UVB radiation (Gidekel et al., 2011). Moreover, the extract has been associated with other properties as the prevention of skin lesions caused by ultraviolet radiation (non melanoma skin cancer) and antineoplasic activity (Gidekel et al., 2009; Pivel et al., 2011).

### SUMMARY OF THE INVENTION

The present invention encompasses a method of preparing a pharmaceutically active extract of D. Antarctica comprising the steps of:
a. Collecting and disinfecting D. Antarctica plants;
b. Exposing the D. Antarctica plants to ultra pure water at a temperature of from 95° to 100° C. for a period of about 5 hours to yield a liquid extract of the plant; and
c. Lyophilizing the liquid extract yielding a dry homogeneous product.

In another embodiment, the present invention provides an anti-aging pharmaceutical composition useful in slowing down or reversing the aging process in human beings said composition comprising an aqueous extract of D. Antarctica. In a particular embodiment the aqueous extract comprises a polyphenolic free-radical scavenger present in the amount of at least 4.0% (w/w) and preferably at least 5.0% (w/w)

In another embodiment the present invention provides a method of treatment for the prevention of photoaging effects generated by exposure to harmful ultraviolet radiation comprising the step of administering to a human being a therapeutically effective amount of the aqueous extract of D. Antarctica. Preferably the extract is administered in the form of a cream, paste or gel.

### BRIEF DESCRIPTION OF THE FIGURES

FIG. 1. Metabolic profile of D. antarctica aqueous extract. The thickened form of peak 1 is probably attributable to the presence of more than 1 compound.

FIG. 2. Chromatographic profile obtained with a semipreparative column, to individually isolate the 11 peaks listed present in the total extract of D. antarctica.

FIG. 3. Relative IL-6 secretion in TNFα-stimulated 3T3-L1 adipocytes treated with D. antarctica whole grass Da(1) and spent material Da (2). Error bars represent 95% confidence intervals.

FIG. 4. Relative IL-6 secretion in TNFα-stimulated 3T3-L1 adipocytes treated with D. antarctica aqueous extract. Values are means of eight observations blocked over two independent experiments. Error bars represent 95% confidence intervals.

FIG. 5. Image of hematoxylin and eosin staining of skin biopsies obtained from the prolonged use test. A and C correspond to the control biopsies before treatment, C and D correspond to biopsies after 60 days of daily treatment with D. antarctic cream at 3%

FIG. 6. AEDA's cytotoxicity on HaCaT cells. Extracts obtained in 2008, 2009, and 2010 were analyzed. The relative proliferation values are shown for the assay without the extract, which shows at 100%. The assays were done by triplicate, and the bars represent the median±SD.

FIG. 7. Effect of different doses of radiation on HaCaT cell viability. The cell viability values are relative to the non-irradiated control. The experiment was carried out in triplicate. All of the points presented ***P<0.001 vs. the control. Each point represents the median±SD.

FIG. 8. The effects of different concentrations of AEDA and ferulic acid on the viability of keratinocytes irradiated with ultraviolet radiation. The cell viability values are relative to the non-irradiated control. The assays were done in triplicate, and the bars represent the median±SD.

FIG. 9. Unsupervised hierarchical clustering of differentially expressed genes. A) Clustering done with all of the genes with a fold change of 1.2 and a P value of 0.05. B) Enlargement of the first segment, where both groups can be seen. UV Control: keratinocytes with ultraviolet radiation. DA-UV: keratinocytes treated with AEDA and ultraviolet radiation.

FIG. 10. Biological processes involving 410 and 747 overexpressed keratinocyte genes treated with AEDA and irradiated with ultraviolet light, versus the control condition, respectively.

FIG. 11. Unsupervised hierarchical clustering of all the experimental conditions. For this clustering, all the differentially expressed genes were taken under all comparison conditions with a P-value of 0.01.

### DETAILS OF THE INVENTION

### 1.—Production of the Aqueous Extract by Temporary Immersion and Separation of Secondary Metabolites

### 1.1. Temporary Immersion System

The Deschampsia antarctica plants (Angiosperm: Poaceae) were collected on the Collins Glacier peninsula (62°22′S, 59°43′W) located in the Antarctic territory. For their establishment in in vitro conditions, the plant material was disinfected with Benomyl and Captan fungicides (1 gL⁻¹) for 20 minutes, then with 40% ethanol for 20 seconds and 10% sodium hypochlorite solution along with Tween 20 for 12 minutes.

To carry out the massive scaling of D. antarctica, the temporary immersion system proposed by Alvard et al. (Alvard and Teisson, 1993) was used, with modifications. The temporary immersion unit consisted of two 4 L glass flasks; one was used to contain the medium and the other for cultivation of the explants. The flasks were connected by a silicone tube that was inserted into the lid of each glass and descended to the bottom, allowing the exchange of culture medium. The system also had a connection through 0.2 μm hydrophobic filters, which guaranteed that the system would remain sterile, while the air pressure was controlled by a gauge and the immersion rate was regulated with a timer, which in turn controlled two three-way solenoid valves, either of which, when opened, allowed air and, consequently, the culture medium to circulate from one container to another. Culture medium in a liquid state was used, based on inorganic salts as proposed by Murashige (1973) (MS), supplemented with 1.0 mgL-1 BAP (Benzylaminopurine N6), sucrose 30 gL⁻¹. The pH of the culture media was adjusted to 5.7 and sterilization was conducted at a pressure of 1.2 kgf·cm⁻² for 20 minutes.

All cultures were kept in growth chambers with a photoperiod of 16 hours light/8 hours dark, with a flow density of photosynthetically active photons oscillating between 1500-2000 μmol m⁻² s⁻¹ and a cultivation temperature of 22±2.0° C.

### 1.2. Obtaining Aqueous Extracts

The initial aqueous extracts of tissues were obtained using a 100 ml (Brand) Soxhlet extractor. For 200 ml of solvent (ultrapure water), 6 grams (±0.05) of dry matter were introduced. The extraction temperature was 95-100° C. and it was controlled with a heating blanket for 5 hours. To obtain a dry, homogeneous product under the conditions to determine the extraction efficiency in dry weight, the aqueous extracts were dried for 48 hours using a lyophilizer (Freezone 4.5, LabConco).

Chestnut-colored aqueous extracts were obtained as a result, with an extraction yield of 38.4% on a dry-matter basis.

1.3. Chemical Composition and Total Polyphenol Content of the D. antarctic Extracts.

The Folin-Ciocalteu colorimetric method (Singleton et al., 1999) can be used to evaluate the total polyphenol content. This technique consists of mixing tungstate and molybdate in a highly basic medium (5-10% Na₂CO₃, aqueous). The polyphenols are easily oxidizable in a basic medium and they react with the molybdate to form molybdenum oxide (MoO). This compound can be identified and quantified by uv/vis spectroscopy because it absorbs at a length of 760 nm.

The total extract was analyzed at a concentration of 100 μg/ml. A calibration curve was made to estimate the total polyphenol content of the samples, according to the absorbance values obtained from known concentrations of gallic acid.

The total D. antarctica polyphenol content is 5.1±0.2%, less than the total polyphenol content in green tea (14-21%) and black tea (8-17%) from Argentina (Anesini et al., 2008), Malaysian green tea (11-14%) and black tea (6-8%) (Chan et al., 2007) and Australian black tea (16%) (Yao et al., 2006).

On the other hand, a complete characterization of the extract was achieved, shown in Table 1. The major components in the extract are salts and sugars. Sugars are found both in the free state (glucose, sucrose, fructose) and joined to flavonoids via ester or ether bonds.

TABLE 1
Physical and chemical composition of the total
aqueous extract of Deschampsia antarctica.

| Parameter | Value |
|---|---|
| Humidity (w/w %) | 2.9 |
| Ash (w/w %) | 11.6 |
| Water solubility (%) | 100 |
| Appearance | Shiny dust |
| Total Sugars (AT) (w/w %) | 81.5 |
| Total Polyphenols (PT) (w/w %) | 5.1 |
| Ratio (AT/PT) | 18.9 |
| Color (Pantone ® Palette) | PMS1405 |

### 1.4. Chromatographic Analysis of the Extract

A reverse-phase HPLC Acme 9000 Youglin Instrument (Vacuum degasser & Mixer, Gradient pump, UV-Vis Detector) was used for a chromatographic analysis of the extracts, with a Kromasil C18 column (250×4.60 mm, particle size of 5 μm) was used. The mobile phase was 0.1% acetic acid (A) and methanol (B) with a gradient of solvent B: 33-50% (17 min), 50% (2 min), 50-33% (5 min) and a flow of 1 mL/min-1 was used. The chromatograms were recorded at a wave length of 362 nm. The injection volume was 20 μL and the total run time was 25 min. The aqueous extract of the tissue was analyzed in situ in a concentration of 1 mgml⁻¹ prepared with ultrapure water.

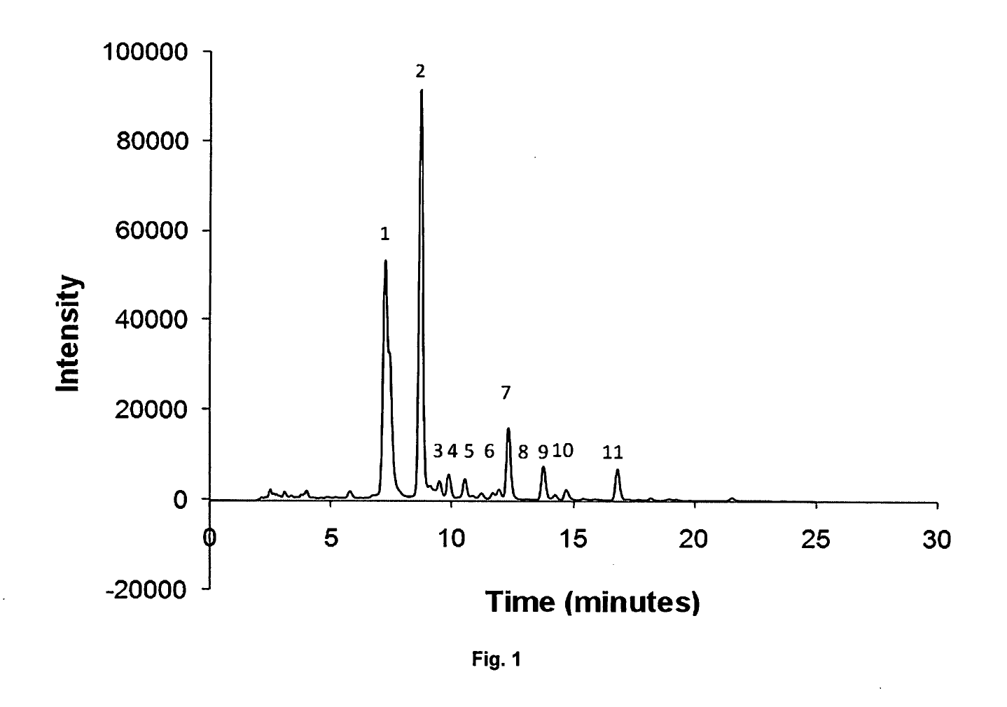

As shown in FIG. 1, 11 symmetrical peaks were obtained under the established chromatographic conditions, resolved at different retention times. However, a shoulder and the width in the first peak suggest that there is more than one metabolite present in it.

1.5. Isolation of the Metabolites from the Extracts

A reverse-phase HPLC Acme 9000 Youglin Instrument (Vacuum degasser & Mixer, Gradient pump, UV-Vis Detector) was used to separate the metabolites present in the extracts, with a Kromasil C18 column (250×100 mm and a particle size 7 of μm), which allowed us to inject a sample of 20 mgL-1. The eluted fractions were collected for their further concentration in a rota evaporator (RV10, IKA) and dried in a lyophilizer (Freezone 4.5, LabConco).

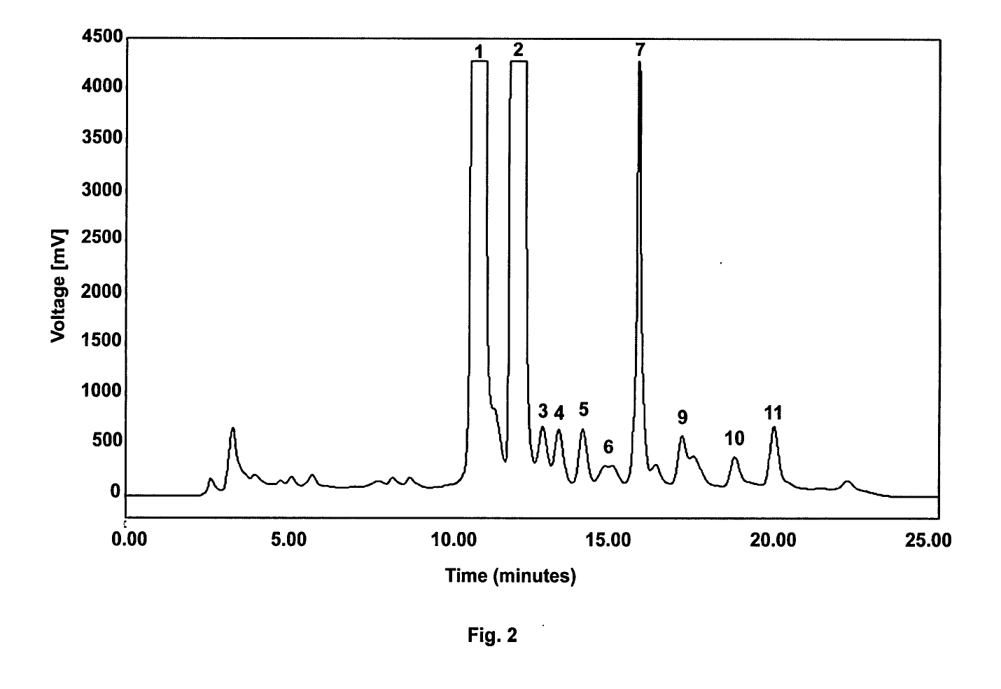

In FIG. 2, we see the chromatographic profile obtained with the semipreparative column. As observed, the metabolic profile of the extract of D. antarctica is preserved, but greater intensities and asymmetries of the peaks are seen, by which the eluted peaks can be identified and collected without error and with greater efficiency.

1.6 Partial Identification of Chromatographic Peaks Using Liquid Chromatography Coupled with Mass Spectrometry (LC-MS)

Later, we decided to perform an identification analysis using mass spectrometry technique so as to amplify our search on the characterization of the 11 defined peaks in the D. antarctica extract. Mass spectrometry is a technology that produces ions in a gaseous phase from organic or inorganic molecules, classifies them depending on their mass/charge (m/z) ratio and measures their intensity. Using this technique, the molecular weight and the abundance of the components in the sample can be determined, to later try to elucidate its structure by banks of masses. For this study, the separation of the D. antarctica extract from in situ tissue was performed in HPLC with the gradients proposed by Van de Staaij et al., (van de Staaij et al., 2002). The collision in the mass spectrometer was defined with parameters previously established in the Department of Metabolomics of Avesthagen in India. In both gradients, the m/z ratio of the ion thrust for each peak ([M+H]⁺ and [MH]⁻) was obtained from which the second fragmentation is obtained and the so-called daughter ions are produced. The molecular weight is determined with the Isilicos Viewer software. The possible structure emerges from a comparison study of precursor and daughter ions, using data obtained from literature.

Table 2 contains the 11 peaks of the total extract of D. antarctica, with their respective ion thrusts that have a positive polarity [M+H]⁺, negative polarity [MH]⁻, daughter ions [MS/MS m/z (ESI^{±})] and the potential candidate compounds of some of them, according to literature.

TABLE 2
Mass spectrometry results for total D. antarctica extract, using the Staaij
elusion gradient. The symbols presented in the table are: t_{R}± retention time, [M + H]⁺
positive polarity ion thrust, MS/MS m/z (ESI⁺), positive polarity daughter ions, [MH]⁻
negative polarity ion thrusts, MS/MS m/z (ESI⁻), negative polarity daughter ions and
characterized compound.

| | | | MS/MS m/z | | MS/MS m/z | |
|---|---|---|---|---|---|---|
| | t_{R}± (min) | [M + H]⁺ | (ESI⁺) | [M − H]⁻ | (ESI⁻) | Compound |
| A | 5.49(+) | 120.12 | | | | |
| A | 8.16(+) | 1072.43 | | | | |
| A | 10.09(+) | 188.06 | | | | |
| A | 16.15(+) | 743.32 | | | | |
| B | 21.71(+) | 163.05 | | | | |
| B | 23.89(+) | 597.31 | | | | |
| B | 25.58(+) | 611.30 | | | | |
| B | 26.55(+) | 177.07 | | | | |
| B | 28.72(+) | 163.03 | | | | |
| B | 29.27(+); 29.33(−) | 611.27 | 449.32; 429.22; | 609.48 | 489.63; 429.53 | 2-O-β |
| | | | 431.57; 413.16; 353.47; | | | galactopyranosyl |
| | | | 329.19; 287.94. | | | orientin |
| | 30.66(+) | 581.28 | | | | |
| 1 | 32.20(+); 32.33(−) | 581.36 | 449.26; 429.48; 431.94; | 579.60 | 357.12 | 2″-O-β |
| | | | 413.37; 383.61; | | | arabinopyranoside |
| | | | 329.15; 299.28 | | | orientin |
| 1 | 33.64(+); 33.95(−) | 449.27 | 431.94; 413.37; 383.61; | 447.70 | | Orientin |
| | | | 353.97; 329.15; | | | |
| | | | 299.28; | | | |
| 2 | 34.81(+); 35.11(−) | 595.35 | | 593.65 | | Isowertiajaponin |
| | | | | | | (7-O- |
| | | | | | | methylorientin 2″- |
| | | | | | | O- |
| | | | | | | arabinopyranosido) |
| 3 | 35.64(+) | 433.23 | | | | |
| 4 | 36.03(+); 36.62(−) | 579.36 | | 577.43 | | |
| 5 | 36.36(+) | 637.35 | | | | |
| 6 | 36.82(+) 37.26(−) | 581.31 | 449.12; 431.31; 413.38; | 579.61 | 459.04; 429.98 | |
| | | | 329.09; 287.32 | | | |
| 7 | 37.30(+); 36.86(−) | 637.36 | | 635.75 | | Isowertiajaponin |
| | | | | | | 2″-O- |
| | | | | | | Arabinopiranosido |
| | | | | | | Acetilado |
| 8 | 38.21(+); 38.61(−) | 679.39 | | 677.78 | | |
| 9 | 39.13(−) | | | 447.35 | | |
| 10 | 39.28(+) 39.61(−) | 563.33 | | 539.79 | | |
| 11 | 40.0(+); 40.34(−) | 679.40 | | 677.67 | | |
| | 40.81(+); 40.96(−) | 663.34 | | 661.72 | | |
| | 45.11(+) | 288.36 | | | | |
| | 49.44(+) | 149.02 | | | | |
| | 50.62(+) | 323.22 | | | | |
| | 52.31(+) | 588.62 | | | | |
| | 54.94(+) | 664.59 | | | | |
| | 56.20(+) | 766.67 | | | | |
| | 56.51(+) | 722.67 | | | | |
| | 59.72(+) | 473.55 | | | | |
| | 70.25(+) | 663.59 | | | | |

These results allow us to confirm the identification made earlier in our laboratories corresponding to orientin 2″-beta-arabinopyranoside (peak 1) and to isoswertiajaponin (7-O-methylorientin) 2″-O-beta-arabinpyranoside (peak 2), both coinciding with those identified by Webby and Markham (Webby and Markham, 1994) as one of the components of D. antarctica. In addition, in this study orientin is added in peak 1 with a molecular ion of 449.27 m/z. (Table 2). Some candidate compounds defined in this study, such as orientin derivatives, have been associated with a photoprotective and antioxidant capacity (Uma Devi, 2001).

### 2. Analysis of the Antioxidant and Anti-Inflammatory Properties of the Extract

Deschampsia antarctica is one of several grass species found on Antarctica's peninsula. Its survival under extreme environmental factors such as low temperature, high light intensity and an increasing UV radiation as result of the Antarctic ozone layer thinning makes it an interesting subject for the study of unique phytochemicals or combinations.

In light of the tremendous cost of type 2 diabetes (T2D), both in terms of human suffering and monetary resources, it is highly desirable to have additional agents to support therapeutic treatment or for the formulation of specialty foods and beverages. Over 400 botanicals have been described for hypoglycemic and antidiabetic use through insulin-like or insulin potentiating action. Further, some botanical products have been shown to improve glucose metabolism and the overall condition of individuals with T2D not only through hypoglycemic effects but also by improving lipid metabolism, antioxidant status, and capillary function.

The objective of this research was to screen three D. antarctica samples for antioxidante and anti-inflammatory activity. Specifically we compared whole D. Antarctica grass Da(1), an aqueous extract DaAE and the spent, post-extracted grass samples Da(2) with respect to (i) free radical quenching of the 2,2′-diphenyl-p-picrylhydrazyl radical (DPPH), and (ii) attenuation of TNFα-stimulated IL-6 secretion.

### 2.1. Antioxidant Activity of D. Antarctica Samples.

### Free Radical Scavenging Activity

Antioxidant activity of the test samples was determined utilizing DPPH, which is a stable radical. The odd electron in the DPPH free radical gives a strong absorption maximum at 550 nm and is purple in color. The color turns from purple to yellow as the molar absorption of the DPPH radical at 550 nm is reduced when the odd electron of DPPH radical becomes paired with hydrogen from a free radical scavenging antioxidant to form the reduced DPPH-H.

The three test samples were dissolved in methanol containing 1% dimethyl sulfoxide and added to microtiter wells in 100 μL aliquots to 100 μL of a 100 μM DPPH solution in methanol to achieve apparent concentrations of 1000, 500, and 100 μg/mL over eight replicates per column in 96-well microtiter plates. Readings were taken at 60 minutes following the addition of the test material. Percent inhibition of the DPPH radical by the test material was computed relative to the inhibition of the DPPH radical by the vitamin E analog Trolox and tabulated as μmol Trolox/g test material. Median inhibitory concentrations (IC50) were also computed.

Of the three samples, only DaAE exhibited antioxidant activity against DPPH (Table 3). No free radical scavenging activity was detected in the two grass samples up to concentrations of 1000 μg/mL. The split aqueous extract samples shipped on two different dates exhibited nearly identical activity of 197 and 190 μmol Trolox/g sample. While the aqueous extracts in our study appeared to be lower in antioxidant activity than the methanol extracted reported in Pereira et al. (Pereira et al., 2009), the 95% confidence interval of the IC50 overlapped the literature value of 295 μg sample/mL indicating potentially no difference (p<0.05) between the samples. As the split samples are identical, further testing used only the labeled “Aqueous Extract Deschampsia antarctica” sample.

A comparison of the antioxidant activity of D. antarctica with 60 commercial botanical products is presented in Table 4. The listed Trolox equivalents of the commercial products are from testing over 400 products in our laboratory and represent those products whose Trolox value falls within the 95% confidence interval of DaAE. Many of the products in this list, such as wild blueberry, raspberry, yerba mate, chrysin and Ligusrum fructus, are known primarily for their antioxidant activity. Pereira attributed the DPPH free radical scavenging activity of the methanol extract of D. antarctica to flavonoids and carotenoids. It is unlikely that the aqueous extract tested in this study contained a significant amount of carotenoids. Flavonoids, although poorly soluble in water, or their glycosides may have contributed to the antioxidant activity seen in the DaAE in this study. Also likely, the antioxidant activity of DaAE could have been due to the presence of the potent antioxidant, phenols hydroxycinnamic acids p-poumaric, caffeic and ferulic acids.

TABLE 3
Median inhibitory concentrations (IC50) and Trolox equivalents in
the DPPH assay of the four Deschampsia antarctica samples.

| | IC50 | Trolox Units (μmol |
|---|---|---|
| Test Material | (μg Sample/mL) | Trolox/g Sample) |
| Deschampsia antarctica | No activity | No activity |
| (1) Ground unextracted | (<1000) | |
| Spent Deschampsia antarctica | No activity | No activity |
| (2) Ground | (<1000) | |
| Aqueous Extract | 474 (258-871)* | 197 (92-417) |
| Deschampsia antarctica | 492 (212-1139) | 190 (82-440) |
| Chilean 1(1) | | |
| Methanol Extract | 295 | 317 |
| Deschampsia antarctica† | | |

*Parenthetic value represents the 95% confidence interval of the estimate.
†Data from (Pereira et al., 2009).

TABLE 4
Relative antioxidant activity of Deschampsia antarctica and 60
commercial products†.

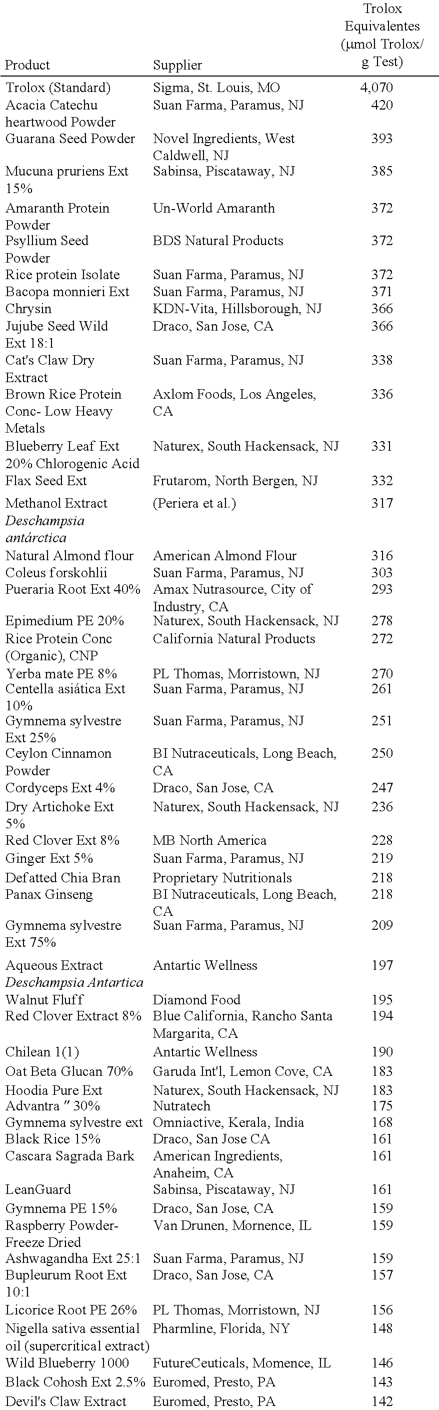

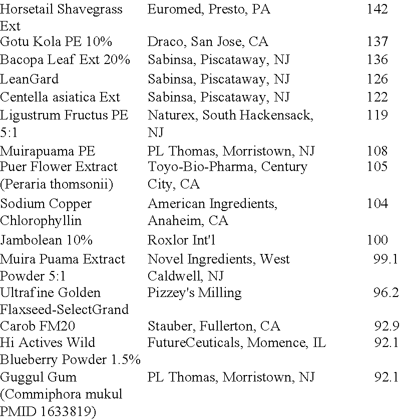

†Products were selected from over 400 tested in our laboratory whose Trolox value
fell within the 95% confidence interval of the D. antarctica aqueous extract sample.

2.2. Anti-Inflammatory Activity of D. antarctica Samples

### Anti-Inflammatory Activity of Test Materials

Post-differentiation, D6/D7 adipocytes were treated with test material 4 h prior to the addition of TNFα at a final concentration of 10 ng/mL. Following overnight incubation of approximately 18 h, the supernatant media were removed and assayed for IL-6.

### IL-6 Cytokine Assay

IL-6 secreted into the medium in response to TNFα stimulation was quantified using the Quantikine® Mouse IL-6 Immunoassay kit Immunoassay kit with no modifications (R&D Systems, Minneapolis, Minn.).

### Statistical Analyses

Experiments assessing the anti-inflammatory activity of the test samples were repeated a minimum of 2 independent times. For statistical analysis, the effects of test materials IL-6 secretion were normalized, respectively, to the TNFα positive control to generate IL-6 indexes. Differences among the treatments were determined by analysis of variance and student's least significant difference test without correction for multiple comparisons; the nominal 5% probability of a type I error was used. Ninety-five percent CI were computed using the ANOVA error mean square estimate of variance (Excel®, Microsoft, Redmond, Wash.).

### TNFα-Stimulated IL-6 Secretion

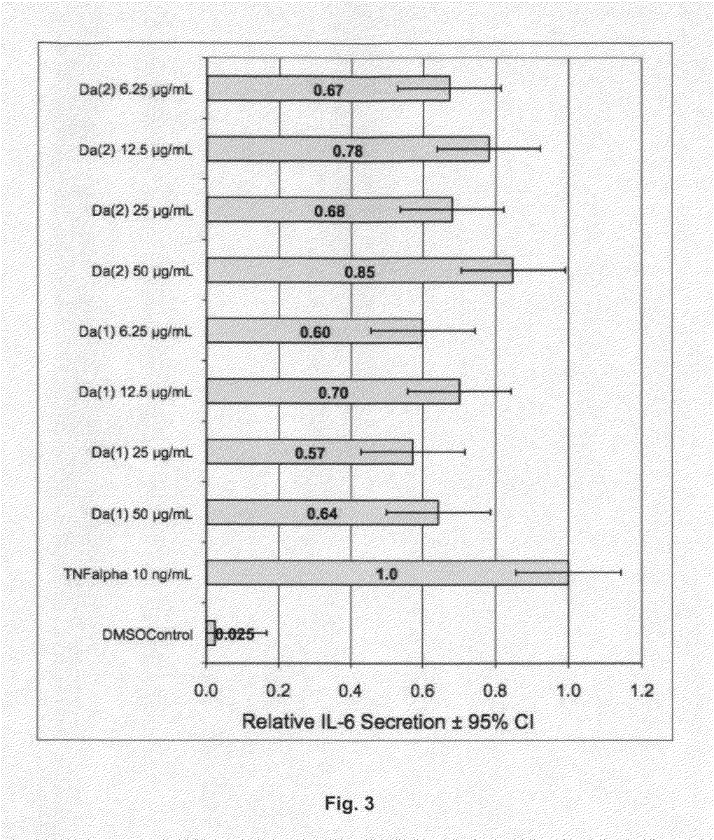

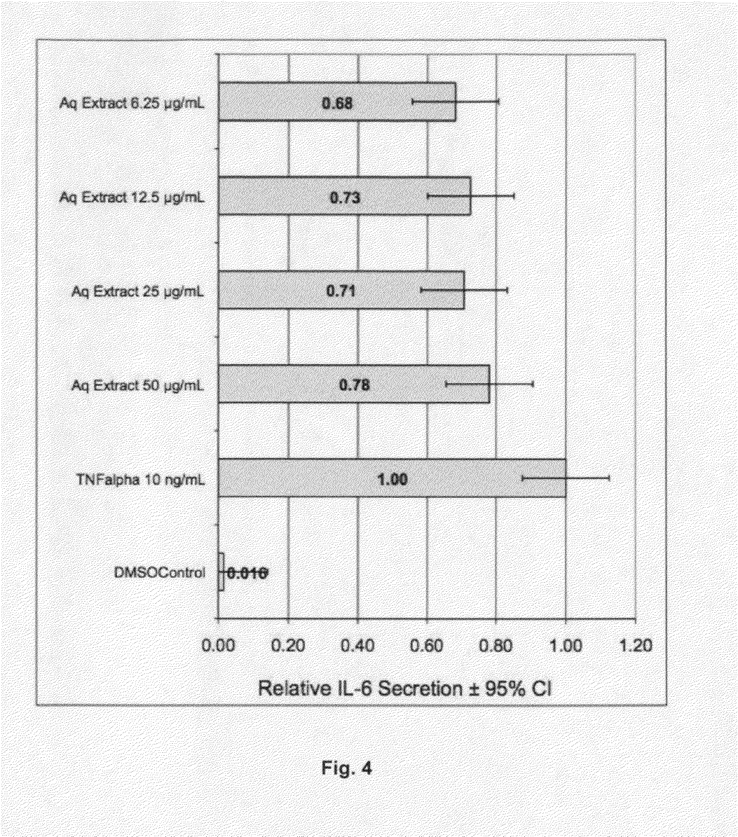

TNFα increased IL-6 secretion from D6/D7 3T3-L1 adipocytes 40- and 63-fold relative to DMSO controls (FIGS. 3 and 4). All three samples attenuated the stimulatory effect of TNFα on IL-6 secretion. At 37% inhibition, the whole grass sample Da(1) was most active, while the spent material Da(2) and DaAE were similar with 26% and 28% inhibition, respectively.

In our final series of experiments, we examined the effect of the three D. antarctica samples on secretion of IL-6 in TNFα-stimulated, mature 3T3-L1 adipocytes. This model was employed to incorporate two common features of T2D, clinically elevated levels of TNFα and adipose tissue dysfunction, into our screening. While failing to inhibit TNFα-stimulated lipolysis, all three D. antarctica samples exhibited substantial anti-inflammatory activity through inhibition of TNFα-stimulated IL-6 secretion. Forskolin increased IL-6 secretion over 100% above the TNFα positive control (data not shown), most likely through its activation of the cAMP-protein kinase A pathway. This discordant response between the D. antarctica samples and forskolin implies a different mechanism of action for D. antarctica on TNFα signaling in the adipocyte that is further supported by our previous observations on lipolysis.

The inhibition of TNFα-stimulated IL-6 secretion by the three D. antarctica samples ranged from 26% to 37% of the positive controls. This level of response was equivalent to rho-iso-alpha acids, cinnamon bark oil, Neem leaves, isoalpha acids, grape seed extract aloe vera PE 40:1 and cinnamon tested in this laboratory. Many of these commercial products have also demonstrated clinical efficacy in T2D and metabolic syndrome.

In the present study, we describe the results of antioxidant and cell-based screening for anti-inflammatory activity of three D. antarctica samples. Based upon the pattern of responses in our battery of assays and information from historical data on the responses of commercial products in these same assays, the probability of a positive in vivo effect on serum fasting glucose or insulin is approximately 82% for DaAE and greater than 62% for Da(1) and Da(2).

### 3. Dermatological Hypoallergenicity Certification Protocol for the Extract and Prolonged Use Test

Patch tests were performed to determine the hypoallergenicity of the products derived from the Deschampsia Antarctica extract in the DA cream formulation at 2% and 3%. In addition, the study was complemented by the “Use Test”, where patients used the product on a regular basis for at least 15 days, and by a prolonged use test.

The D. Antarctica cream was made in the Quality Assurance laboratory of Laboratorios BagÓ in Chile. The extract concentration was homogenized in an aqueous hypoallergenic base.

### 3.1. Patch Test Protocol

The studies were conducted in groups of 21 people over 18, using aluminum chambers (Finn Chambers) (according to Dermatotoxicology Methods: The Laboratory Worker's Vademecum, Marzulli-Maibach, 1998) containing approximately 0.5 gr of product. The procedure consists of applying the patch on the subject's back for 48 hours and removing it at the end of that period. The procedure's effects are evaluated immediately after administering and at 48 and 96 hours.

The following method was employed to read the patch:

(−): no reaction

(*): irritative reaction

(+): erythema, edema in 50 percent of the patch's surface

(++): papules, vesicles in 50 percent of the patch's surface

(+++): papules, vesicles over 50 percent of the patch's surface

Tables 4a, b contains the records of the volunteer patients that participated in the study and the results obtained from the Patch and Use Tests. We can conclude from the results that the DA product, both at 2% and 3%, demonstrated, after the dermatological tests were done, that it is non-irritating and meets hypoallergenicity conditions.

TABLE 5
Description of the volunteers who participated in the
study and the main results of the patch test and use test
for Deschampsia cream at 2% (A) and 3% (B).

| Patient | Age | | Reaction time test | | |
|---|---|---|---|---|---|
| (Number) | (year) | Sex | 48 h | 96 h | Use test result |

A

| | | | | | |
|---|---|---|---|---|---|
| 1 | 22 | F | (−) | (−) | (−) |
| 2 | 41 | F | (−) | (−) | (−) |
| 3 | 33 | F | (−) | (−) | (−) |
| 4 | 62 | F | (−) | (−) | (−) |
| 5 | 21 | F | (−) | (−) | (−) |
| 6 | 22 | M | (−) | (−) | (−) |
| 7 | 25 | F | (−) | (−) | (−) |
| 8 | 34 | F | (−) | (−) | (−) |
| 9 | 31 | F | (−) | (−) | (−) |
| 0 | 22 | F | (−) | (−) | (−) |
| 11 | 42 | M | (−) | (−) | (−) |
| 12 | 34 | F | (−) | (−) | (−) |
| 13 | 46 | M | (−) | (−) | (−) |
| 14 | 50 | M | (−) | (−) | (−) |
| 15 | 52 | F | (−) | (−) | (−) |
| 16 | 18 | M | (−) | (−) | (−) |
| 17 | 18 | M | (−) | (−) | (−) |
| 18 | 50 | M | (−) | (−) | (−) |
| 19 | 53 | F | (−) | (−) | (−) |
| 20 | 34 | M | (−) | (−) | (−) |
| 21 | 25 | F | (−) | (−) | (−) |

B

| | | | | | |
|---|---|---|---|---|---|
| 1 | 22 | F | (−) | (−) | (−) |
| 2 | 41 | F | (−) | (−) | (−) |
| 3 | 33 | F | (−) | (−) | (−) |
| 4 | 62 | F | (−) | (−) | (−) |
| 5 | 21 | F | (−) | (−) | (−) |
| 6 | 22 | M | (−) | (−) | (−) |
| 7 | 25 | F | (−) | (−) | (−) |
| 8 | 34 | F | (−) | (−) | (−) |
| 9 | 31 | F | (−) | (−) | (−) |
| 0 | 22 | F | (−) | (−) | (−) |
| 11 | 42 | M | (−) | (−) | (−) |
| 12 | 34 | F | (−) | (−) | (−) |
| 13 | 46 | M | (−) | (−) | (−) |
| 14 | 50 | M | (−) | (−) | (−) |
| 15 | 52 | F | (−) | (−) | (−) |
| 16 | 18 | M | (−) | (−) | (−) |
| 17 | 18 | M | (−) | (−) | (−) |
| 18 | 50 | M | (−) | (−) | (−) |
| 19 | 53 | F | (−) | (−) | (−) |
| 20 | 34 | M | (−) | (−) | (−) |
| 21 | 25 | F | (−) | (−) | (−) |

### 3.2. Prolonged Use Test

The prolonged use test consisted of continuous topical application to the ankle region of the volunteers. We proceeded to perform skin biopsies (3 mm diameter skin punch) in 5 volunteers before starting application of the product and at 2 months into the treatment.

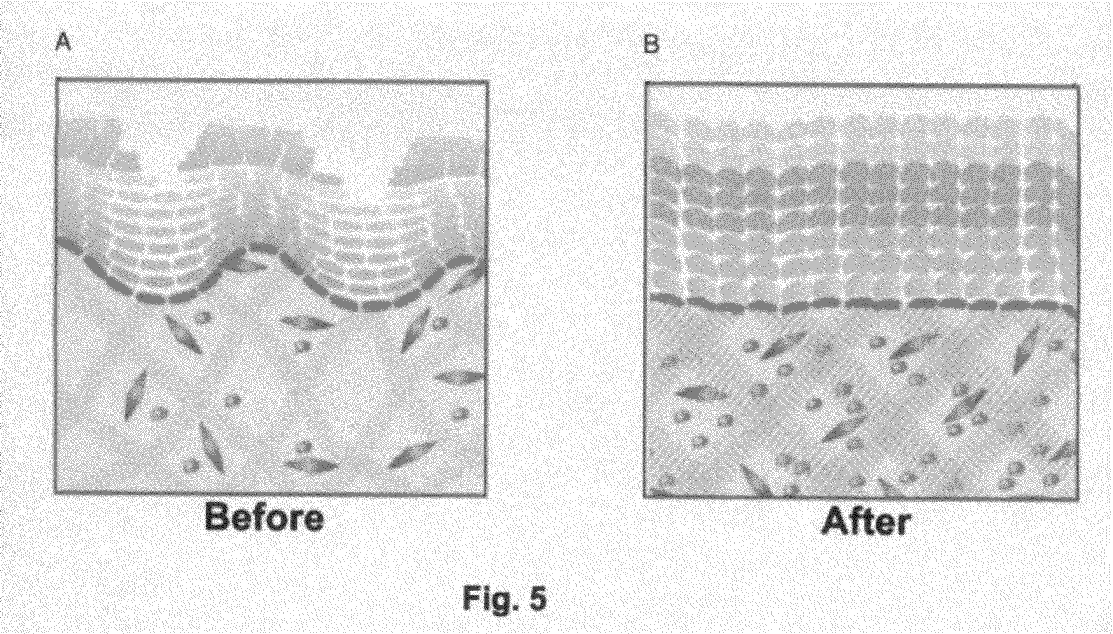

As shown in FIG. 5, when performing the microscopic examination, both samples of skin with attachments showed no morphological evidence of cutaneous aging. Comparatively, the sample obtained 2 months after topical therapy shows a higher ordering of the keratinocytes in the epidermal layer, with an increase in the granular layer and a decrease of the stratum corneum. In the dermis, a greater number of capillary blood vessels are seen, without major changes in the reorganization and arrangement of the collagen-elastic fibers (signed by Dr. F. Chavez-Rojas, Dermatologist. Anatomic Pathologist).

### 4. Molecular Mechanism of Action of the Extract by Microarray Technology

4.1 Study of the Cytotoxicity of Aqueous Extract of Deschampsia antarctica on Human Keratinocyte Cells (HaCaT)

The study of the cytotoxic effect of AEDA was carried out on human keratinocyte cell lines. The extracts used in the trial were obtained from Deschampsia antarctica plants gathered in 2008, 2009, and 2010.

The human keratinocyte lines, or HaCaT, were cultured in a high glucose DMEM medium supplemented with 10% fetal bovine serum (FBS) (Hyclone, Logan, Utah, USA) and 100 U/mL of penicillin plus 100 μg/mL streptomicine (Gibco BRL, Gaithersburgh, Md., USA). Once the culture reached confluence, 5,000 cells were seeded in a total volume of 100 ul in 96-well plates. The cells were then incubated during 24 hours at 37° C. in an atmosphere of 5% CO₂ and 90% humidity. After incubation, descending concentrations of AEDA were added to each well. The cells with extract were incubated for 72 hours, and the cell viability was then tested in a MTS assay.

MTS Assay to Measure Cell Viability

The MTS assay uses a colormetric method to determine the number of viable cells in proliferation. The assay uses the MTS solution (a tetrazolium compound) and a PMS electron coupling reagent. The cells bioreduce the MTS to form a formazan product, whose absorbance can be measured at 490 nm. The MTS is converted to soluble formazan by dehydrogenase enzymes found in the cell's active metabolic processes. The amount of formazan product measured to 490 nm will be directly proportionate to the number of live cells in the culture.

After 72 hours of incubation with the extract, the cells were washed with PBS to eliminate saturation in the reading due to the intensity of the extract's color. The cells were incubated for 3 hours with a solution of 100 ul of medium and 20 ul of the MTS solution in each well. Finally, the absorbance was determined in a plate spectrophotometer at 490 nm.

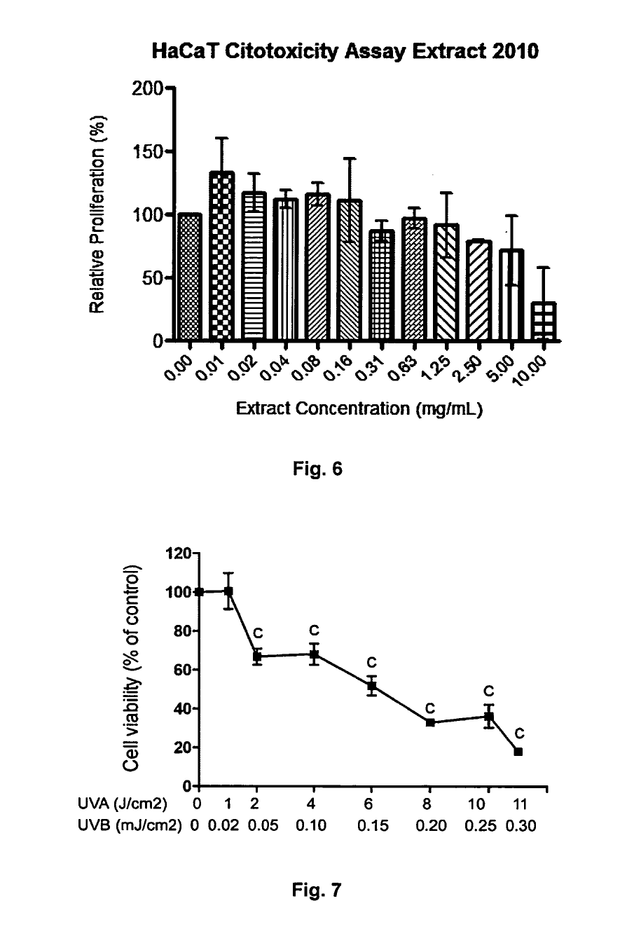

The FIG. 6, contains three graphs that show the results of the aqueous extract of D. antarctic (AEDA) cytotoxicity assay on HaCaT cells. At concentrations of 10 mg/ml and 5 mg/ml, the extract has a cytotoxicity of 29-49% and 50-70%, respectively. Based on these results, it was decided to use concentrations of less than 5 mg/ml in subsequent photoprotection experiments.

4.2. Effect of Aqueous Extract of Deschampsia Antarcticaon Human Keratinocyte Cells (HaCaT) with the Application of UVA and UVB Radiation

To evaluate the intensity of the UVA and UVB light with which the HaCaT cells with the extract would be irradiated. First, the effect of different intensities of radiation on cell viability was determined.

For this assay, 100,000 cells were seeded in a 24-well plate at a total volume of 500 ul in a complete culture medium. After incubating for 24 hours at 37° C. in an atmosphere of 5% CO₂, the cells were irradiated with different concentrations of UV light in an irradiation system inside a type 2 biosafety laminar flow hood. The system consisted of 2 florescent tubes, one UVA-emitting 36 W PL-L (320-400 nm) and one UVB-emitting 20 W TL (290-320 nm) (Philips, Eindhoven, the Netherlands). Both tubes were located 5 cm from the bottom of the well to produce homogeneous irradiation intensity. The irradiation intensity was monitored with radiometers for UVB (Solarmeter 6.2 digital UVB radiometer, Solartech Inc., Harrison Township, Mich.) and UVA (Tecpel, CHY-732, Taiwan). The HaCaT cells were irradiated at different time intervals (Table 5) to reach the different accumulative doses of UVA and UVB (Gao et al. 2007). After irradiation with UVA+UVB light, the cells were incubated for 24 hours and the viability of the cells was measured with the MTS assay.

The FIG. 7 shows that with increased ultraviolet irradiation, cell viability decreases depending on the dose. When the ultraviolet radiation dose for UVA+UVB was 4 J/cm²+0.1 mJ/cm² and 6 J/cm²+0.15 mJ/cm², cell viability decreased 68% and 51%, respectively. Based on these results, we decided to work with UVA+UVB radiation levels of 4 J/cm²+0.1 J/cm².

TABLE 5
Curve done for different intensities of UVA + UVB irradiation.
At a distance of 5 cm from the bottom of the plate, the homo-
geneous radiation intensity was 4.42 mJ/s for UVA and 1.35 mJ/s
for UVB. Based on this intensity, an irradiation time with
UVA + UVB was estimated to generate the specific accumulative
intensity.

| | Time (min) | | | | | | | |
|---|---|---|---|---|---|---|---|---|
| | 3.80 | 7.50 | 15.00 | 22.00 | 30.0 | 37.70 | 36.70 | 41.50 |
| UVA Radiation | 1.00 | 2.00 | 4.00 | 6.00 | 8.00 | 10.00 | 9.75 | 11.00 |
| (J/cm²) | | | | | | | | |
| | Time (min) | | | | | | | |
| | 0.25 | 0.61 | 1.20 | 1.90 | 2.50 | 3.00 | 9.30 | 12.30 |
| UVB Radiation | 0.02 | 0.05 | 0.10 | 0.15 | 0.20 | 0.25 | 0.75 | 1.00 |
| (J/cm²) | | | | | | | | |

Once a specific radiation intensity was selected for the experiment, the effect of incubating the keratinocytes with different concentrations of AEDA was determined (previously set in the cytotoxicity assays).

In this assay, 100,000 cells were seeded in a 24-well plate. After incubating for 24 hours at 37° C. in an atmosphere of 5% de CO₂, the cells were washed with PBS and incubated with AEDA doses of between 4.5 and 0.5 mg/ml previously diluted in PBS. After three hours of incubation with AEDA, we used a control group (without irradiation) and a group that was irradiated with ultraviolet light at 4 J/cm² UVA and 0.1 J/cm² UVB. After irradiation, 500 ul of complete culture medium was added to each well, and 24 hours later the MTS assay was performed to determine cell viability. Doses of 0.5-2.5 nM of ferulic acid were used as a control. Ferulic acid is a chemical compound with high antioxidant activity that is isolated from the plant cell wall. It acts as a protective shield and inhibits damage to DNA.

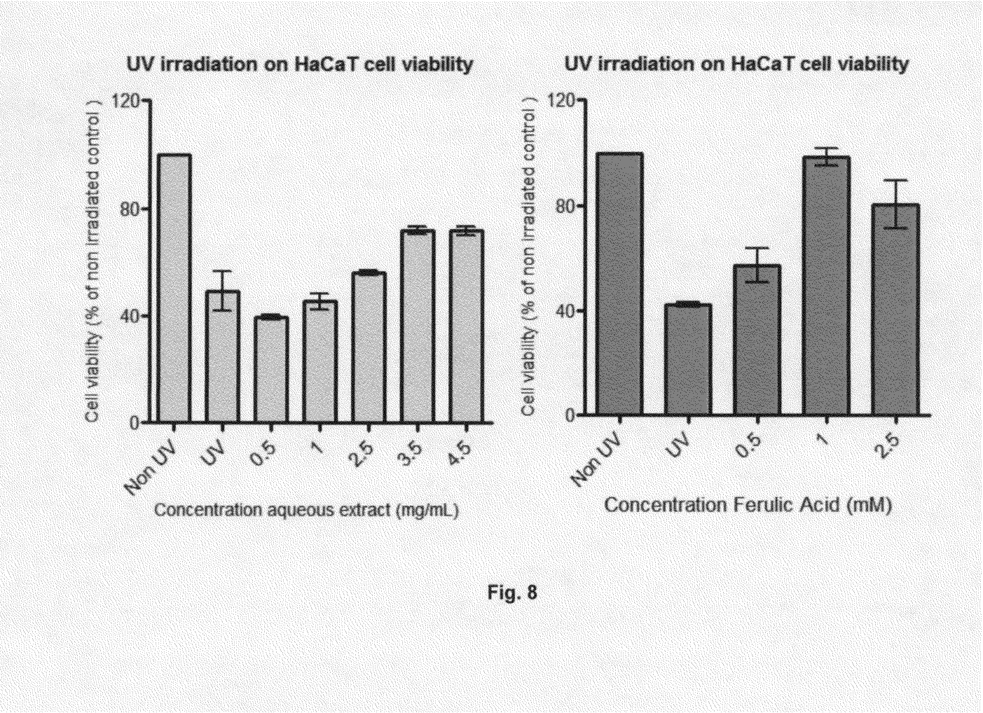

As shown in FIG. 8, higher concentrations of AEDA are directly related to increased viability of the irradiated keratinocyte cells. With AEDA concentrations of 3.5 and 4.5 mg/ml, we see cell viability increase to 72%. A ferulic acid concentration of 1 mM holds cell viability at 100%; higher doses present a possible photocytotoxic effect on the cells (which was not found in the non-irradiated control, results not shown). Based on these results, the dose was set at 3.5 mg/ml of AEDA and 1 mM of ferulic acid for subsequent research on AEDA's action mechanisms.

4.3. Study of the Molecular Mechanism of the Action of Aqueous Extract of Deschampsia antarctica on Keratinocytes Irradiated with UVA and UVB Light.

The carcinogenic effects of ultraviolet radiation can be decreased through apoptosis, or programmed cell death, and the elimination of the damaged DNA or mutated cells. Microarrays genes expression studies have shown that UVA and UVB radiation causes expression changes in keratinocytes; these changes have led to a greater understanding of the impact of UV radiation on keratinocytes at the molecular level, especially the mechanism associated with apoptosis (He et al., 2004; Lee et al., 2005). Various changes at the molecular level have been associated with aging and cancer caused by high exposure to ultraviolet rays, as exposure to UV radiation triggers a stream of ROS and inflammatory keratinocytes by activating the AP-1 and NF-κB pathways.

In this study, we used microarray technology to identify genes and functional enrichments that may help us to understand how it prevents the carcinogenic and photoaging effects of ultraviolet radiation

As in the previous assays, the cells were seeded on 24-well plates and left to grow for 24 hours. Next, the cells were washed with PBS, and each row (consisting of 4 wells) was incubated with 3.5 mg/ml of AEDA and 1 mM of ferulic acid; PBS alone was used as a negative control. After 3 hours of incubation, we used a control group (without irradiation) and a group that was irradiated with a dose of 4 J/cm² of UVA and 0.1 J/cm² of UVB. After the irradiation, 500 ul of complete culture medium was added to each well and incubated for 24 hours. To confirm the biological effect of each treatment, MTS was used to analyze the cell viability in one of the wells, as described above.

Prior to extracting the RNA, the culture medium was removed from each well and washed twice with cold PBS. The E.Z.N.A.® Total RNA Kit I (Omega Bio-Tek, Georgia, USA) was used for RNA extraction, following the supplier's instructions. In addition, the protocol described by the same supplier, E.Z.N.A.®Total RNA DNase Digestion Protocol, was used to eliminate traces of genomic DNA. The RNA's concentration and purity was determined by UV spectrophotometry (NanoDrop Technologies, USA), and its integrity was verified by 1% agarose gel electrophoresis under denaturing conditions.

### Microarray Labeling, Hybridization, and Analysis

The slides used for hybridization were 48.5k HEEBO (Human Exonic Evidence-Based Oligonucleotide, Microarray Inc., Huntsville, USA) developed by Stanford University and Illumina. These slides consist of a set of 39,000 probes that codify for exonic sequences and splicing options.

HEEBO microarrays are two color chips allowing the hybridization of two samples per chip. In this case, a reference design was used where a Universal Human Reference (UHR, Clontech, Palo Alto, Calif., USA) where labeled with Alexa 555 and aRNA from cells with and without irradiation, treated with AEDA and ferulic acid where labeled with Alexa 647. Under this design each sample where hibridizaded on each microarray and reference against with UHR.

Amplification and labeling was carried out with the SuperScript Indirect RNA Amplification System (Invitrogen, Carlsbad, Calif., USA) commercial kit according to the supplier's instructions. Used as a template for each reaction was 1 μg of total RNA taken from human keratinocyte cells treated with AEDA and ferulic acid, with and without UV irradiation.

The hybridization probe was 60 pmoles of universal reference aRNA labeled with Alexa Fluor 555 and 60 pmoles of the aRNA from the experimental condition labeled with Alexa Fluor 647 in a final volume of 50 ul with 1× hybridization buffer (5×SSC, 50% formamide, SDS 0.1%, salmon sperm DNA). This mixture was incubated for 2 minutes at 95° C. and applied to the slide previously prehybridized with 5×SSC, 0.1% SDS, and 0.1% BSA for 30 minutes at 50° C. Hybridization was carried out in a wet chamber with 2×SSC (Inslide Out, Boekel Scientific, Pennsylvania, PA, USA) for 16 hours at 42° C. After hybridization, the slides were washed once for 5 minutes at 42° C. with 2×SSC and 0.1% SDS, for 5 minutes at room temperature with 0.1×SSC and 0.1% SDS, and twice for 1 minute with 0.1×SSC. Finally, the slides were centrifugally dried and scanned in a ScanArray Gx (PerkinElmer, Wellesley, Mass., USA).

The signal intensity of the slides was quantified with SpotReader software (Niles Scientific, USA), and the data was analyzed with Limma R (www.r-project.org) (free available at www.bioconductor.org) The microarray intensity data was background corrected (normexp methods) (Ritchie et al., 2007). Then printiploess method was used for within-slide normalization and the scale method was used for between slide normalization.

After normalization, two pathways between samples were hierarchically clustered using Euclidean distance as metric similarity criteria, and average linkage was the clustering method, using the MeV program: MultiExperiments Viewer, which is part of the TM4 Microarray Software Suite (http://www.tm4.org/mev/).

Results

To elucidate the photoprotective effect of aqueous extract of D. Antarctica, we used oligonucleotide microarray technology, a methodology through which we can obtain the transcriptome, or all of the mRNA expressed by the cell under a specific treatment. This study was carried out on the HaCaT keratinocyte cell line, which is a cell line with differentiation similar to normal keratinocytes and that is used as an in vitro model to understand the molecular events underlying malignant transformations in epithelial cells after exposure to UVA or UVB rays. To begin this analysis, we focused on investigating the molecular response to UVA and UVB radiation in the human keratinocyte cell line in the control condition. We then observed the difference in gene expression in the extract-treated cells exposed to radiation, and finally we studied whether the molecular action mechanism was similar to or different from that of ferulic acid.

In comparing the gene expression between the control condition of keratinocytes irradiated with UVA and UVB versus non-irradiated cells, we found a total of 1276 genes that expressed differently in irradiated keratinocytes, 781 of which were overexpressed genes and 495 were repressed. In order to understand the genes that were altered by UVA and UVB exposure, the genes were classified according to their biological function (Table 6). There are various functional categories altered by exposure to UVA and UVB, particularly the activation of various genes involved in the cell apoptosis cycle, junctional proteins in the cytoskeleton, cell metabolism and growth regulation, response to extracellular matrix stimulation, aging, and response to reactive oxygen species. The genes have the same behavior in all of these functional categories, which indicates the presence of biological processes that are affected in their entirety by UVA and UVB radiation. Important among the genes activated by ultraviolet exposure are those involved in apoptosis, such as the activation of caspase 8, which cleaves caspase 3 and 7 and activates the pro-apoptotic cascade, and various apoptosis-facilitating genes such as BCL2L11, TRAF4, STAT1, TP53, etc. An important example is the c-Jun gene, a proto-oncogene that overexpresses when the MAPK pathway is activated by radiation, with the h-ras-JNK-c-Jun-AP1 signaling axis a precursor to malignant transformation and aging (Choi et al., 2009).

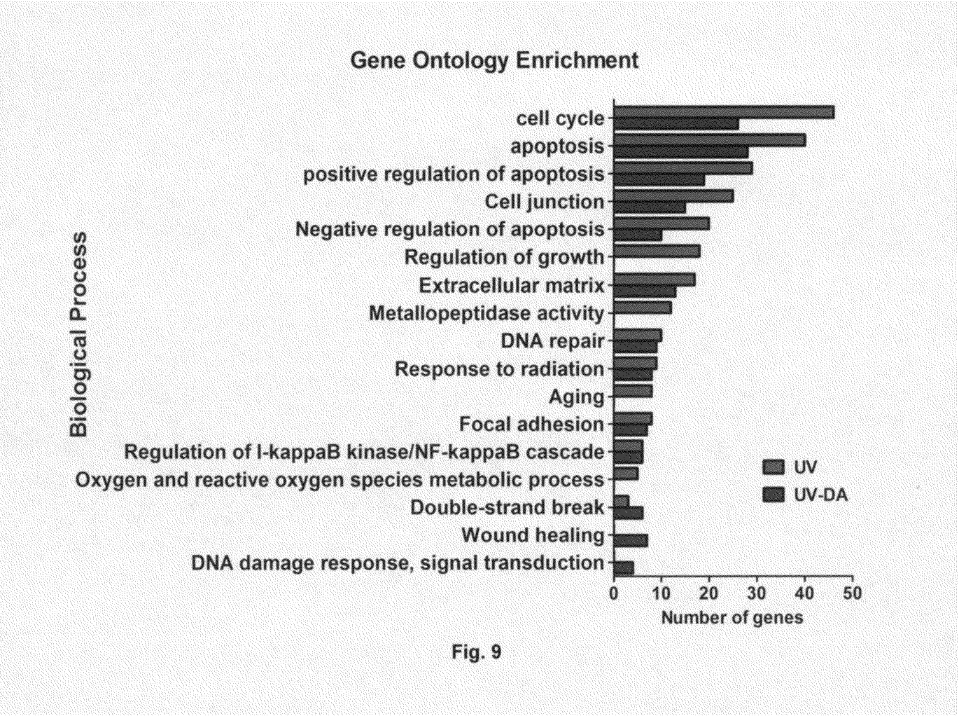

When comparing the differential gene expression profile of keratinocyte genes treated with AEDA against the control condition, under ultraviolet radiation in both cases, a total of 1130 differentially expressed genes were found. Of these, 410 were overexpressed genes and 710 were repressed, when compared to the keratinocytes that had been treated with the extract. FIG. 9 shows that with unsupervised hierarchical clustering, these differential genes are able to discriminate between the two groups: AEDA-treated keratinocytes (DA-UV) and control condition (UV control), both under ultraviolet exposure.

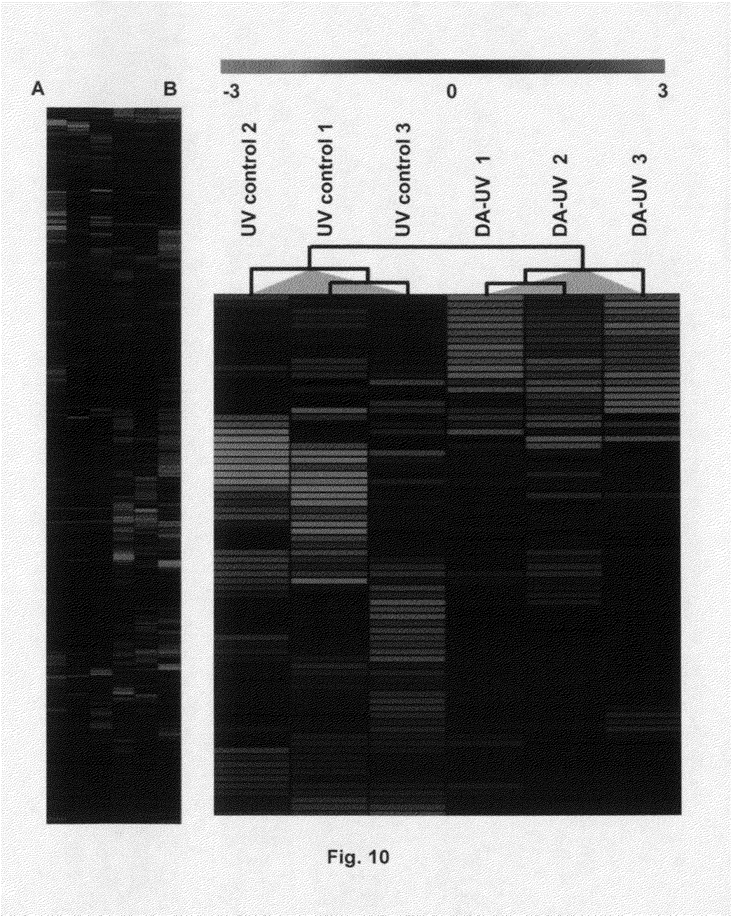

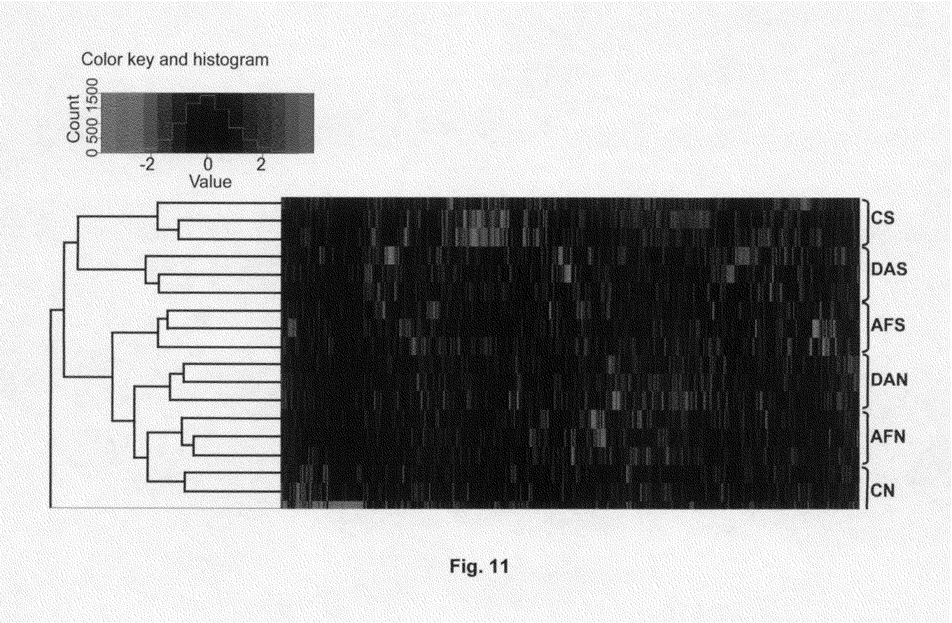

A comparison of the overexpressed genes involved in each functional category under the following comparisons: keratinocytes treated with irradiated D. antarctica vs. the control treated with irradiated PBS, and control keratinocytes treated with irradiated PBS vs. the non-irradiated control (FIG. 10). Again we see that the main biological functions involved in the keratinocytes' response to high UVA and UVB radiation are the cell cycle, apoptosis, junctional proteins in the cytoskeleton, regulation of cell metabolism and growth, response to extracellular matrix stimulation, aging, and response to reactive oxygen species. Meanwhile, we see a decrease in genes involved in regulating apoptosis and the cell cycle among the keratinocytes irradiated with UVA and UVB and treated with D. antarctica extract, along with an increase in genes that belong to other functional categories, such as the DNA damage response and wound healing. Table 7 shows the differentially expressed genes that are overexpressed and repressed among the keratinocytes treated with AEDA vs. the control condition. To assess how the extract behaves in relation to other experimental conditions, such as the control cells and those treated with ferulic acid, with and without ultraviolet radiation, an assessment was made of the genes that show different behavior when exposed to different treatments. For this analysis, we worked with the high quality slide “spots,” with a value in all the experimental slides and a p-value of <0.01.

Subsequently differential genes obtained from the different comparisons were clustered: “CS v/s DAS”, “CS v/s AFS”, AFS v/s DAS″, “AFN v/s DAN”, “CN v/s DAN”, “CN v/s AFN”. CS: control with ultraviolet; DAS: Deschampsia antarctica extract with ultraviolet; AFS: Ferulic acid with ultraviolet; CN: non-irradiated control; DAN: Deschampsia antarctica non-irradiated extract and AFN: non-irradiated ferulic acid. As shown in FIG. 11, in this clustering we can see that the conditions under ultraviolet radiation and with AEDA are quite different from each other. But what is striking is that the AEDA-treated condition is quite different from the condition treated with ultraviolet and ferulic acid, which predicts that the two compounds have different action mechanisms. Also, the conditions without ultraviolet radiation are clustered together, and the distances are near ferulic acid, demonstrating the action of this compound in absorbing and blocking ultraviolet radiation.

With these results, it is proposed that AEDA reduces the damage caused by exposure to ultraviolet light by activating the DNA-repairing genes that protect cells from apoptosis. Important among the overexpressed genes found in keratinocytes exposed to high UV radiation and treated with AEDA are: the EYA1 gene (Eyes absent homolog 1), a tyrosine phosphatase that dephosphorylates the Tyr142 from the H2AX histone, which plays a major role in repairing DNA after exposure to ionizing radiation (Cook et al., 2009); hMSH2 (DNA mismatch repair protein MSH2), which plays an important role in protecting against tumorigenesis caused by UVB radiation and preventing skin cancer (Young et al., 2003); and BRCA1, which plays an essential role in three different complexes to repair DNA damage:homologous recombinational repair (HRR), non-homologous end joining, and nucleotide excision repair (NER). BRCA1 carries out these functions by interacting with components of the DNA repair machinery and by regulating the expression of genes that are involved in DNA repair pathways, associating the absence of BRCA1 with an accumulation of chromosome damage, cell cycle abnormalities, and apoptosis, leading to the development of abnormalities and tumorigenesis (Deng and Wang, 2003). Other relevant genes that are overexpressed in the AEDA-treated condition are: LIG3 and APTx (aprataxin), which make up part of the base excision repair complex (BER); RPA3, which makes up part of the nucleotide excision repair complex (NER) (Wood et al., 2001); and RAD51 and RAD52, member genes of the non-homologous repair complex that interact with BRCA1 to help repair damaged DNA through the HRR pathway (Deng and Wang, 2003).

TABLE 6
Microarray analysis of gene expression alterations in HaCaT cells subjected to high ultraviolet radiation
(UVA + UVB). The differentially expressed genes, both overexpressed and repressed, were grouped according to the most
representative functional categories. The 1276 genes from the irradiated and non-irradiated control cell comparison were
used to construct the table, with a fold change level of >1.2 and a P-value of <0.05

| Biological | | | P- | | | |
|---|---|---|---|---|---|---|
| Process | Up-regulated Genes | Count | value | Down-regulated genes | Count | P-value |
| Cell cycle | ZWINT, REC8, NUP37, SEPT4, NEDD9, | 46 | 0.010 | EID1, PRC1, DBF4, MLH3, TTN, | 30 | 0.047 |
| | NUSAP1, HGF, CDC7, DNM2, CHFR, ITGAE, | | | URGCP, LLGL2, APP, MACF1, | | |
| | CDK1, CCNA1, TUBE1, AIF1, MAP9, RNF8, | | | THBS1, VCPIP1, STAG1, ESCO1, | | |
| | PSME3, CDCA3, CYLD, TTN, KCTD11, CDK2, | | | ANAPC2, PDS5B, CGRRF1, | | |
| | PPP1R15A, HBP1, TBRG4, ABL1, MAP3K8, | | | MKI67, MSH2, STRADA, GAS2, | | |
| | TP53, C13ORF15, MAD2L2, CLIP1, ALG11, | | | TPD52L1, CDKN3, ATM, RNF8, | | |
| | UTP14C, MNS1, NEK2, CUL4A, KIAA0892, | | | MFN2, SASS6, NSL1, CENPV, | | |
| | PKHD1, NCAPD3, MYH10, CCNT2, PRC1, | | | ADAM17, G0S2 | | |
| | IRF6, HAUS2, MAPK1 | | | | | |
| Apoptosis | JUN, FIS1, CCAR1, HRAS, CASP8, CSE1L, | 40 | 0.003 | RTN4, ING3, CCL2, TNFSF18, | 26 | 0.259 |
| | CASP7, TICAM1, MEF2A, SCRIB, STAT1, | | | APP, BDNF, RARB, THBS1, LTB, | | |
| | BNIP1, CSRNP1, DAD1, GADD45B, STK4, | | | TOP2A, RAB27A, BRAF, DFFA, | | |
| | FGD1, PSME3, SQSTM1, BAG1, KALRN, | | | MSH2, ADAMTS20, GRIN1, ATM, | | |
| | BCL2L11, LTBR, TRAF4, SOD2, BID, | | | STK3, DAPK1, TNFSF13B, P2RX1, | | |
| | PPP1R15A, CASP4, TBRG4, ACVR1C, | | | RASGRF1, F2, ADAM17, ID3, IL2 | | |
| | NDUFA13, LOC100134381, ARHGEF9, | | | | | |
| | DOCK1, TP53, TIAL1, DEDD2, ITGB2, ELMO1, | | | | | |
| | SGK1, BFAR | | | | | |
| Response to | JUN, SOD2, PEMT, ATG1611, STAT1, PCSK9, | 18 | 0.008 | IL20RB, OSMR, F2, GRIN1, | 11 | 0.011 |
| extracellular | ALDOB, COX4I1, OXT, CD44, STC2, TP53, | | | ADAM17, CCL5, THBS1, CPB2, | | |
| stimulus | DAD1, TSHB, CYP11A1, LEP, RPS19, | | | TMPRSS6, PLAU, IL2 | | |
| | RPS19P3, RASGRP4 | | | | | |
| Cell junction | MLF2, SCRIB, NEDD9, DNM2, CD44, DOK7, | 25 | 0.364 | ENAH, LIMS2, GLRA1, S100A7, | 25 | 0.002 |
| | CLDN11, STARD8, RABAC1, PVRL4, SHANK1, | | | SYT6, ABI1, TENC1, APBB1IP, | | |
| | PANX2, ASH1L, ARHGEF7, EPB41L5, DSG3, | | | WNT2, PTK2, GPHN, SORBS1, | | |
| | NRAP, SHROOM2, ZNF384, LIMS2, PKHD1, | | | PVRL1, MACF1, CHRNA5, SCN5A, | | |
| | SYNM, CHRNB4, PGM5, CADPS2 | | | NPHP1, GRIN1, CHRM5, LAMA3, | | |
| | | | | ADAM17, VAMP3, UNC13C, ADD3, | | |
| | | | | LCP1 | | |
| Extracellular | CD248, PCSK6, MMP3, ELN, ADAMTS20, | 17 | 0.410 | FRAS1, MATN3, ADAMTS20, | 13 | 0.138 |
| matrix | TNC, CRTAP, ADAMTS12, CD44, FBLN1, | | | MMP26, WNT2, LAMA1, LAMA4, | | |
| | LAMC1, ADAMTS2, TIMP2, EFEMP1, GPLD1, | | | WNT4, LAMA3, IMPG1, THBS1, | | |
| | FBN2, PI3 | | | COL11A2, USH2A | | |
| Regulation of I- | CD40, CASP8, TICAM1, SQSTM1, GOLT1B, | 6 | 0.439 | SLC44A2, TLR3, TMEM189, LTB | 4 | 0.489 |
| kappaB kinase/NF- | LTBR | | | | | |
| kappaB cascade | | | | | | |
| Negative regulation | CSF2, HRAS, NOS3, SOD2, ADAMTS20, HGF, | 20 | 0.131 | CCL2, BRAF, MSH2, ADAMTS20, | 14 | 0.163 |
| of apoptosis | BNIP1, SYVN1, PCSK6, RXFP2, TP53, DAD1, | | | DFFA, GRIN1, TNFSF18, ATM, | | |
| Negative regulation | CSF2, HRAS, NOS3, SOD2, ADAMTS20, HGF, | 20 | 0.131 | CCL2, BRAF, MSH2, ADAMTS20, | 14 | 0.163 |
| of apoptosis | BNIP1, SYVN1, PCSK6, RXFP2, TP53, DAD1, | | | DFFA, GRIN1, TNFSF18, ATM, | | |
| | CDK1, CITED2, NME2, NME1-NME2, NME1, | | | DAPK1, BDNF, TNFSF13B, | | |
| | SELS, SQSTM1, BFAR, BAG1, SON | | | ADAM17, THBS1, IL2 | | |
| Positive regulation | JUN, CASP8, TICAM1, STAT1, SCRIB, PCSK9, | 29 | 0.009 | | | |
| of apoptosis | BNIP1, TNFRSF8, DNM2, CD44, STK4, FGD1, | | | | | |
| | SQSTM1, KALRN, BCL2L11, BID, ARHGEF7, | | | | | |
| | CD2, CASP4, WWOX, NDUFA13, ABL1, | | | | | |
| | LOC100134381, ARHGEF9, TP53, SMPD1, | | | | | |
| | TIAL1, DEDD2, CUL4A, MAPK1 | | | | | |
| Histone | SUV420H1, ASH1L, WHSC1, WHSC1L1, | 6 | 0.021 | | | |
| methyltransferase | FBXO11, PRMT8 | | | | | |
| activity | | | | | | |
| Double-strand break | SOD2, RNF8, TP53 | 3 | 0.723 | | | |
| repair | | | | | | |
| Release of | JUN, CASP7, SOD2, BID, TP53 | 5 | 0.009 | | | |
| cytochrome c from | | | | | | |
| mitochondria | | | | | | |
| Aging | JUN, HRAS, NOS3, SOD2, SLC6A3, ROMO1, | 8 | 0.159 | | | |
| | MORC3, TP53 | | | | | |
| Gluconeogenesis | PCK2, G6PC3, PCK1, ALDOB | 4 | 0.079 | | | |
| Response to | | | | RNF8, APP, REV1, TRPC3, CCL2, | 10 | 0.092 |
| radiation | | | | MSH2, GRIN1, SDF4, ATM, NPHP1 | | |
| Tumor necrosis | | | | TNFSF13B, TNFSF18, LTB | 3 | 0.106 |
| factor receptor | | | | | | |
| binding | | | | | | |
| Ubiquitin-dependent | | | | ANAPC2, USP8, UBE3A, UBE2K, | 6 | 0.786 |
| protein catabolic | | | | SMURF1, EDEM1 | | |
| process | | | | | | |
| DNA repair | | | | POLL, RNF8, MORF4L1, ESCO1, | 11 | 0.237 |
| | | | | RFC4, REV1, MSH2, MLH3, | | |
| | | | | TOP2A, ATM, RPA3 | | |
| | | | | POLL, MORF4L1, ESCO1, ATG10, | | |
| | | | | REV1, MSH2, TAOK3, MLH3, ATM, | | |

TABLE 7
Microarray analysis of gene expression alterations in HaCaT cells treated with AEDA and subjected to high
ultraviolet radiation (UVA + UVB). The differentially expressed genes, both overexpressed and repressed, were grouped
according to the most representative functional categories. The 1130 genes from the irradiated and non-irradiated control
cell comparison were used to construct the table, with a fold change level of >1.2 and a P-value of <0.05.

| Biological | | | | | | |
|---|---|---|---|---|---|---|
| Process | Up-regulated genes | Count | P-Value | Down-regulated genes | Count | P-Value |
| Apoptosis | PRF1, ADORA2A, RTKN, DNAJB13, | 28 | 0.003 | TNFRSF6B, CSF2, TNFRSF8, | 31 | 0.007 |
| | ZNF346, APLP1, APP, MAP3K5, CSE1L, | | | GPX1, CASP4, BAG1, CD44, | | |
| | LTA, TRAF3, BCL10, ABR, MSH2, SMAD3, | | | CASP8, AATF, TRAF7, WWOX, | | |
| | KIAA1967, GAS2, FADD, BRCA1, STK3, | | | RTEL1, IL1A, ADAM9, IL6, | | |
| | TRAF3IP2, PLEKHF1, BAX, RIPK1, TIAL1, | | | ADAMTS20, ARHGEF7, | | |
| | RHOT2, PDCD7, THOC1 | | | TGFBR1, SKP2, ARHGEF9, | | |
| | | | | STAT1, BCL2L11, BCL2L10, | | |
| | | | | MAPK1, ATF5, SON, JUN, | | |
| | | | | SMPD1, APAF1, ABL1, | | |
| | | | | UNC13B, DNM2 | | |
| Regulation of I- | TRAF3IP2, BCL10, RIPK1, UBE2V1, | 6 | 0.097 | LTBR, CASP8, UBE2V1, | 5 | 0.239 |
| kappaB kinase/NF- | FADD, LTA | | | NDFIP2, CD40 | | |
| kappaB cascade | | | | | | |
| Cell junction | JUB, S100A7, PKHD1, ARHGAP17, | 15 | 0.323 | GJB7, NRAP, DSG3, DSG1, | 7 | 0.263 |
| | ACTN3, VCL, P2RX4, PRUNE, LAMA3, | | | CLDN5, CRB3, ADD3 | | |
| | PVRL1, CD44, RASGRP2, VAMP3, | | | | | |
| | HTR3A, NPHP1 | | | | | |
| Histone | SUV420H2, EHMT1, MLL2 | 3 | 0.263 | WHSC1L1, MLL3, SUV420H1 | 3 | 0.234 |
| methyltransferase | | | | | | |
| activity | | | | | | |
| Cell cycle | JUB, SPIN1, ZAK, PKHD1, PKMYT1, | 26 | 0.120 | CCNT2, FZR1, AIF1, NEK2, | 36 | 1.33E+11 |
| | MLH3, TTN, CCNG2, LATS2, LLGL2, APP, | | | ITGAE, TTN, UHMK1, CYLD, | | |
| | CAMK2D, TXNL4B, ANAPC2, CGRRF1, | | | TSPYL2, MACF1, HSPA2, | | |
| | MSH2, LIG3, SMAD3, GAS2, RAD52, | | | MAP3K8, CAMK2D, MNS1, | | |
| | BRCA1, RAD51, MCM6, RASSF1, | | | NUP37, RAD51L3, CCNA1, | | |
| | GAS2L2, RUVBL1 | | | CDC7, KIAA0892, ANAPC4, | | |
| | | | | LIG3, SKP2, ILF3, GAS7, | | |
| | | | | DCTN1, MIS12, MAPK1, | | |
| | | | | EIF4G2, ZWINT, NEK9, CHFR, | | |
| | | | | ABL1, DST, KCTD11, UTP14C, | | |
| | | | | DNM2 | | |
| Response to | CUBN, ASNS, UGT1A1, UGT1A7, | 7 | 0.477 | LEP, GPX1, IL6, EDN1, TAC1, | 7 | 0.167 |
| extracellular | UGT1A10, UGT1A6, UGT1A9, UGT1A8, | | | SCGB1A1, CXCL12 | | |
| stimulus | CD44, UGT1A5, STC1, LTA, MEST | | | | | |
| Negative regulation | BCL10, EYA1, DLX1, ADORA2A, MSH2, | 10 | 0.530 | TNFRSF6B, CSF2, IL6, | 13 | 0.125 |
| of apoptosis | BAX, SMAD3, SOX4, ASNS, RASA1 | | | ADAMTS20, TGFBR1, SKP2, | | |
| | | | | BCL2L10, ATF5, GPX1, SON, | | |
| | | | | BAG1, AATF, IL1A, RTEL1 | | |
| DNA repair | EYA1, MSH2, LIG3, APTX, MLH3, RAD52, | 9 | 0.422 | TNFRSF6B, CHD1L, RAD23A, | 7 | 0.652 |
| | BRCA1, RAD51, RPA3 | | | POLD2, LIG3, RAD51L3, ABL1, | | |
| | | | | RTEL1 | | |
| Ubiquitin-dependent | ANAPC2, UBE3A, UCHL5, USP46 | 4 | 0.945 | USP7, USP28, CYLD, FZR1, | 11 | 0.057 |
| protein catabolic | | | | UBE2K, RAD23A, ANAPC4, | | |
| process | | | | SKP2, FBXO4, CHFR, UBE2L3 | | |
| Extracellular matrix | MATN3, LTBP1, TNXB, EMILIN3, WNT2B, | 13 | 0.109 | | | |
| | APLP1, LGALS3BP, LAMA3, CD44, | | | | | |
| | EMID1, COL12A1, PRSS36, ADAMTS2 | | | | | |
| Positive regulation | BCL10, ABR, ZAK, ADORA2A, SMAD3, | 19 | 0.024 | | | |
| of apoptosis | SOX4, FADD, BRCA1, STK3, PLEKHF1, | | | | | |
| | APP, MAP3K5, CD44, BAX, RIPK1, TIAL1, | | | | | |
| | LTA, PDCD7, TRAF3 | | | | | |
| Double-strand break | EYA1, MSH2, APTX, RAD52, BRCA1, | 6 | 0.020 | | | |
| repair | RAD51 | | | | | |
| Response to | APP, EYA1, ZAK, HMGCR, MSH2, BAX, | 8 | 0.239 | | | |
| radiation | BRCA1, NPHP1 | | | | | |
| DNA damage | ZAK, MSH2, MAPK14, BRCA1 | 4 | 0.326 | | | |
| response, signal | | | | | | |
| transduction | | | | | | |
| Wound healing | JUB, FOXA2, CD44, ADORA2A, HPS6, | 7 | 0.348 | | | |
| | SMAD3, SCNN1B | | | | | |
| Cellular response to | | | | TNFRSF6B, RAD23A, LIG3, | 12 | 0.772 |
| stress | | | | GPX1, MAPK1, CHD1L, ADM, | | |
| | | | | JUN, POLD2, AATF, RAD51L3, | | |
| | | | | ABL1, RTEL1 | | |
| Fatty acid metabolic | | | | LEP, LPL, PLP1, C5ORF4, | 6 | 0.495 |
| process | | | | EDN1, HSD17B4 | | |
| Regulation of | | | | GPX1, IL6, TAC1, SCGB1A1 | 4 | 0.260 |
| inflammatory | | | | | | |
| response | | | | | | |
| Regulation of | | | | GPX1, APAF1, STAT1, | 4 | 0.279 |
| caspase activity | | | | BCL2L10 | | |

### REFERENCES

Alberdi, M., Bravo, L. A., Gutierrez, A., Gidekel, M., and Corcuera, L. J. (2002). Ecophysiology of Antarctic vascular plants. Physiol Plant 115, 479-486.
Alvard, D., and Teisson, C. (1993). Comparison of methods of liquid medium culture for banana micropropagation. Plant Cell 32, 55-60.
Barcikowski A, Lyzwtnska R, and Zarzycki K (1999). Growth rate and biomass production of Deschampsia antarctica Desv. in the Admirality Bay region. South Shetland Islands Antarctica. Polish Polar Research 20, 301-311.
Bravo, A., L., Ulloa, Nancy, Zuñiga, E., G., Casanova, Angelica, Corcuera, J., L., et al. (2001). Cold resistance in Antarctic angiosperms, Vol 111 (Oxford, ROYAUME-UNI, Blackwell).
Bravo, L., and Griffith, M. (2005). Characterization of antifreeze activity in Antarctic plants. J Exp Bot 56, 1189-1196.
Bystrzejewska, G. (2001). Photosynthetic temperature response of antarctic plant Deschampsia antarctica and of temperate region plant Deschampsia coespitosa. Polish Journal of Ecology 49, nr 3, 215-219.
Cook, P. J., Ju, B. G., Telese, F., Wang, X., Glass, C. K., and Rosenfeld, M. G. (2009). Tyrosine dephosphorylation of H2AX modulates apoptosis and survival decisions. Nature 458, 591-596.
Corte, A. (1961). Fertilidad de las semillas fanerógenas que crecen en el Cabo Primavera (Costa de Danco), Peninsula Antártica. Contr Inst Antarct Argentino. 65, 1-16.
Chan, E., Lim, Y., and Chew, Y. (2007). Antioxidant activity of Camellia sinensis leaves and tea from a lowland plantation in Malaysia. Food Chemistry 102, 1214-1222.
Choi, H. S., Bode, A. M., Shim, J. H., Lee, S. Y., and Dong, Z. (2009). c-Jun N-terminal kinase 1 phosphorylates Myt1 to prevent UVA-induced skin cancer. Mol Cell Biol 29, 2168-2180.
Day, T., Ruhland, C., and Xiong, F. (2001). Influence of solar ultraviolet-B radiation on Antarctic terrestrial plants: results from a 4-year field study. J Photochem Photobiol B 62, 78-87.
Deng, C. X., and Wang, R. H. (2003). Roles of BRCA1 in DNA damage repair: a link between development and cancer. Hum Mol Genet 12 Spec No 1, R113-123.
Gidekel, M., Molina, R., Cabrera, G., Sunkel, C., Gutierrez, A., Pivel, J., Ferrer, J., and Sanz, M. (2011). Agent for cutaneous Photoprotection Against UVA/UVB Rays.
Gidekel, M., Weber, H., Cabrera, G., Gutierrez, A., Osorio, J., Becera, J., Podhajcer, O., Cafferata, E., Sunkel, C., and Mihovilovic, I. (2009). New extracts of Deschampsia antarctica desv. with antineoplastic activity.
He, Y. Y., Huang, J. L., Sik, R. H., Liu, J., Waalkes, M. P., and Chignell, C. F. (2004). Expression profiling of human keratinocyte response to ultraviolet A: implications in apoptosis. J Invest Dermatol 122, 533-543.
Holderegger, R., Stehlik, I., Smith, L., and Abbott, R. J. (2003). Populations of Antarctic hairgrass (Deschampsia antarctica) show low genetic diversity. Artic Antarctic and Alpine Research 35, 214-217.
Lee, K. M., Lee, J. G., Seo, E. Y., Lee, W. H., Nam, Y. H., Yang, J. M., Kee, S. H., Seo, Y. J., Park, J. K., Kim, C. D., et al. (2005). Analysis of genes responding to ultraviolet B irradiation of HaCaT keratinocytes using a cDNA microarray. Br J Dermatol 152, 52-59.
Lewis Smith, A. R. (2003). The enigma of Colobanthus quitensis and Deschampsia antarcica in Antarctica. In: Huickes A H I, Gieskes W W C, Schorno R L M, van der Vies S M, Volff W I. Antarctic biology in a global context, 234-239.
Pereira, B. K., Rosa, R. M., da Silva, J., Guecheva, T. N., Oliveira, I. M., Ianistcki, M., Benvegnu, V. C., Furtado, G. V., Ferraz, A., Richter, M. F., et al. (2009). Protective effects of three extracts from Antarctic plants against ultraviolet radiation in several biological models. J Photochem Photobiol B 96, 117-129.
Pivel, J., Ferrer, J., Gidekel, M., Molina, R., Cabrera, G., Sunkel, C., Gutierrez, A., Juarranz, J., Espada, J., and Sanz, F. (2011). Uso de una composición que contiene un extracto de una planta gramínea para la prevención de lesiones cutáneas originadas por la radiación ultravioleta.
Ritchie, M. E., Silver, J., Oshlack, A., Holmes, M., Diyagama, D., Holloway, A., and Smyth, G. K. (2007). A comparison of background correction methods for two-colour microarrays. Bioinformatics 23, 2700-2707.
Ruhland, C. T., and Day, T. A. (2001). Size and longevity of seed banks in Antarctica and the influence of ultraviolet-B radiation on survivorship, growth and pigment concentrations of Colobanthus quitensis seedlings. Environ Exp Bot 45, 143-154.
Singleton, V., Orthofer, R., and Lamuela-Raventos, R. (1999). Analysis of total phenols and other oxidation substrates and antioxidants by means of folin-ciocalteu reagent Methods in Enzymology 299, 152-178.
Uma Devi, P. (2001). Radioprotective, anticarcinogenic and antioxidant properties of the Indian holy basil, Ocimum sanctum (Tulasi). Indian J Exp Biol 39, 185-190.
van de Staaij, J., de Bakker, N. V., Oosthoek, A., Broekman, R., van Beem, A., Stroetenga, M., Aerts, R., and Rozema, J. (2002). Flavonoid concentrations in three grass species and a sedge grown in the field and under controlled environment conditions in response to enhanced UV-B radiation. J Photochem Photobiol B 66, 21-29.
Wang, S. Q., Balagula, Y., and Osterwalder, U. (2010). Photoprotection: a review of the current and future technologies. Dermatol Ther 23, 31-47.
Webby, R. F., and Markham, K. R. (1994). Isoswertiajaponin 2″-O-beta-arabinopyranoside and other flavone-C-glycosides from the Antarctic grass Deschampsia antarctica. Phytochemistry 36, 1323-1326.
Wood, R. D., Mitchell, M., Sgouros, J., and Lindahl, T. (2001). Human DNA repair genes. Science 291, 1284-1289.
Yaar, M., and Gilchrest, B. A. (2001). Ageing and photoageing of keratinocytes and melanocytes. Clin Exp Dermatol 26, 583-591.
Yaar, M., and Gilchrest, B. A. (2007). Photoageing: mechanism, prevention and therapy. Br J Dermatol 157, 874-887.
Yao, L., Jiang, L., Caffin, N., D'Arcy, B., Datta, N., Liu, X., and Singanusong, R. (2006). Phenolic compounds in tea from Australian supermarkets. Food Chemistry 96, 614-620.
Young, L. C., Hays, J. B., Tron, V. A., and Andrew, S. E. (2003). DNA mismatch repair proteins: potential guardians against genomic instability and tumorigenesis induced by ultraviolet photoproducts. J Invest Dermatol 121, 435-440.
Zuñiga, G., Alberdi, M., and Corcuera, L. (1996). Non-structural carbohydrates in Deschampsia antarctica Desv. from South Shetland Islands, Maritime Antarctic. Environmental and Experimental Botany 36, 393-398.

1. A method of preparing a pharmaceutically active aqueous extract of D. Antarctica comprising the steps of:
a. Collecting and disinfecting D. Antarctica plants;
b. Exposing the D. Antarctica plants to ultra pure water at a temperature of from 95° to 100° C. for a period of about 5 hours to yield a liquid extract of the plant; and
c. Lyophilizing the liquid extract followed by separation of a dry homogeneous product.

2. A pharmaceutical composition for slowing down or reversing the aging process in human beings comprising an aqueous extract of D. Antarctica according to claim 1.

3. A pharmaceutical composition for slowing down or reversing the aging process according to claim 2 wherein said aqueous extract further comprises a polyphenolic free-radical scavenger.

4. A pharmaceutical composition for the prevention of photoaging effects in human beings generated by exposure to harmful ultraviolet radiation comprising an aqueous extract of D. Antarctica according to claim 1.

5. A method of treatment for slowing down or reversing the aging process in human beings comprising administering a therapeutically effective amount of an aqueous extract of D. Antarctica according to claim 1.

6. A method of treatment for the prevention of photoaging effects in human beings generated by exposure to harmful ultraviolet radiation comprising administering a therapeutically effective amount of an aqueous extract of D. Antarctica according to claim 1.

7. A pharmaceutical composition according to claims 3 and 4 wherein said composition is in the form of a cream, paste or gel.

8. An aqueous extract of D. Antarctica comprising a polyphenolic free-radical scavenger present in the amount of at least 4.0% (w/w).
